(19) United States
(12) Patent Application Publication
(10) Pub. No.: US 2011/0264122 A1
(43) Pub. Date: Oct. 27, 2011

(54) DEVICE FOR APPLYING GLUE ON TISSUES TO BE CONNECTED

(76) Inventors: Mauro Bonino, Torino (IT); Guido Rusco, Chieri (Torino) (IT)

(21) Appl. No.: 13/128,707

(22) PCT Filed: Nov. 6, 2009
(86) PCT No.: PCT/IB2009/054946

§ 371 Date: Jul. 12, 2011

(30) Foreign Application Priority Data
Nov. 11, 2008 (IT) TO2008A000828

(51) Int. Cl. A61B 17/11

(52) U.S. Cl. 606/153

(57) ABSTRACT

A device for applying a connecting glue (1) to the facing extremities (T1, T2) of two portions of tissue to be connected, comprising a diffusing element (3) suppliable with the glue (C) and interposable between the two portions of tissue (T1, T2) to be connected, wherein the diffusing element (3) is provided with openings (10) for bilateral delivery of the glue (C) towards the two portions of tissue to be connected in anastomosis.

The present invention relates to a device for applying connecting glue on tissues to be connected, for example, on tissues to be sutured in anastomosis.

Anastomosis generally intends the sutured connecting of the walls of two hollow viscera so to make them communicating. Anastomoses are frequent in gastrointestinal surgery, but they may involve any hollow organ, from the biliary to urinary tract, and blood vessels.

With reference to anastomoses, and, in particular, intestinal anastomoses, a distinction is usually made among termino-terminal anastomoses, wherein two stumps are arranged end to end, termino-lateral anastomoses wherein a terminal end is sutured to the lateral wall (suitably opened) of another viscera or vice versa and latero-lateral anastomoses when two stumps are arranged side by side and anastomised after opening two incisions of suitable length.

The formation of a dehiscence or enteric fistula is a very severe complications associated with sutured anastomoses that occur with a frequency ranging from 10 to 20% in subjects subjected to intestinal anastomosis, wherein the term dehiscence indicates the spontaneous reopening of a previously sutured wound and the term enteric fistula indicates a communication between two structures or cavities of the organism allowing the exit of gas or liquids, for example, enteric liquids. The dehiscence of a surgical anastomosis constitutes a severe complication and is the main cause of death in gastroenteric resection surgery. An intestinal anastomotic dehiscence causes, in fact, mainly the free diffusion of enteric material into the abdominal cavity, with consequent generalized peritonitis or generation of peri-anastomotic infective processes.

The patent application WO-A-2006/044799 describes a device for applying glue for intestinal anastomoses providing, in particular, extra-luminal application of the glue, that is, on the trunks of already sutured intestine. The device is provided with a main channel, inside of which conduits for conveying the glue are present, and two arms having the capacity of opening and closing, wrapping around the anastomosis site so to apply the glue material directly on the previously sutured tissue.

However, such device does not solve the problem of the formation of dehiscence since the connecting glue is applied on the outer membrane of the sutured tissue, wherein such membrane, known as the serous membrane due to it specific composition does not allow activation of the healing mechanisms that prevent the onset of dehiscence and/or fistulae.

The present invention has the object of providing a device allowing minimisation and virtual elimination of the risks of onset of dehiscence and/or fistulae, preferably anastomotic dehiscence and/or enteric fistulae.

According to the present invention, such object is achieved by means of the solution specifically recalled in the claims that follow. The claims form an integral part of the technical teaching provided herein relative to the invention.

One embodiment of the solution described herein is a device for applying a connecting glue to the facing extremities of two portions of tissue to be connected by enteric anastomosis performable with a mechanical suturer, wherein the device comprises a diffusing element suppliable with glue and interposable between the two portions of tissue to be connected, wherein the diffusing element is provided with openings for the bilateral delivery of the glue towards the two portions of tissue to be connected. The diffusing element also has a deployable structure to cooperate with the above-said mechanical suturer, as will be described below.

The present inventors have verified that the application of a connecting glue to the facing extremities of the two portions of tissue before connecting them, for example by anastomosis, allows minimisation of the risk of the formation of dehiscence or fistulae. The application of connecting glue to the tissue portions before connecting them (manually or by means of a suturing machine) allows, in fact, acceleration of the tissue healing processes, reduction of peri-operative blood loss, and, consequently, reduction in the morbidity induced by accumulation of blood or other fluids in the tissues neighbouring the surgical wound (delayed healing, infections, etc.). In fact, due to its structural characteristics, the device described herein allows the connecting glue to reach and coat also the muscular layer and mucosal layer, which being highly vascularised allow the activation of the healing mechanism driven by the interaction of plasma components such as thrombin, fibrinogen, fibronectin and factor XIII, and of growth factors.

An additional advantage obtainable with the application of the connecting glue onto the facing portions of tissue before they are sutured (manually or by means of a suturing machine) is provided by a notable improvement in the distribution of tensions between the portions of tissue successively connected. For example, in the case in which a fibrin-based connecting glue is employed, the glue polymerises three-dimensionally, forming a fibrin network that—by means of peptide bonds—binds to terminal amino acids on the cells constituting the tissue portions to be connected and causes resistance to traction in the order of 750 grams per square centimetre between the two portions of tissues once they are connected. In the case of gastrointestinal anastomoses, such fibrin veil between the two portions of tissue to be connected will contribute—with no increase in thickness—to the uniform distribution of suture tensions. Such improvement in the distribution of suture tensions also allows reduction or notable attenuation of the appearance of ischemic points with a consequent reduction in tissue necrosis phenomena in correspondence with the sutures. In this way, the connecting glue provides an element complimentary to the action of the suture stitches, metallic or not.

In the case of connection by anastomosis, the connecting glue may be applied also between the virtual opposing spaces of the suture stitches, metallic and not, and on the cut edge of the muscular layer and of the mucosal layer when operating with a mechanical suturer.

The invention will now be described in detail, by way of non-limiting example, with reference to the annexed figures, wherein:

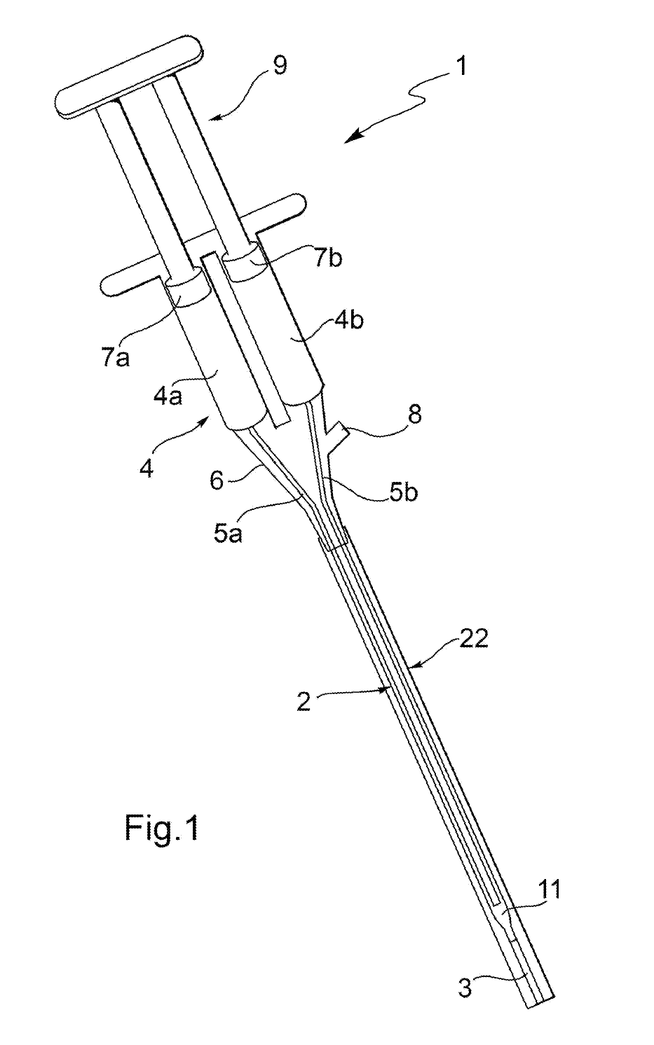

FIG. 1 illustrates a view in section of one embodiment of a device for applying glue as described herein;

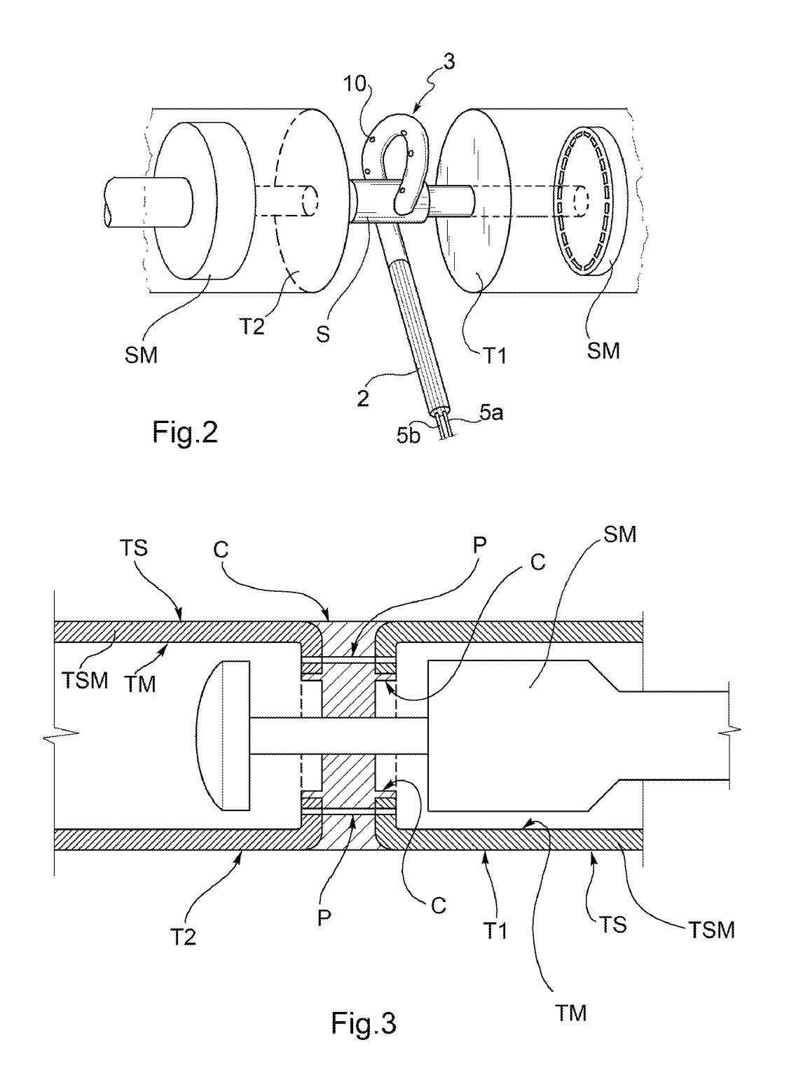

FIG. 2 illustrates a prospective view of the distal portion of the device for applying glue being used on an intestinal anastomosis;

FIG. 3 illustrates a view in longitudinal section of two lumen trunks, to which a connecting glue has been applied by means of the device for applying glue described herein, during anastomosis with a mechanical suturer;

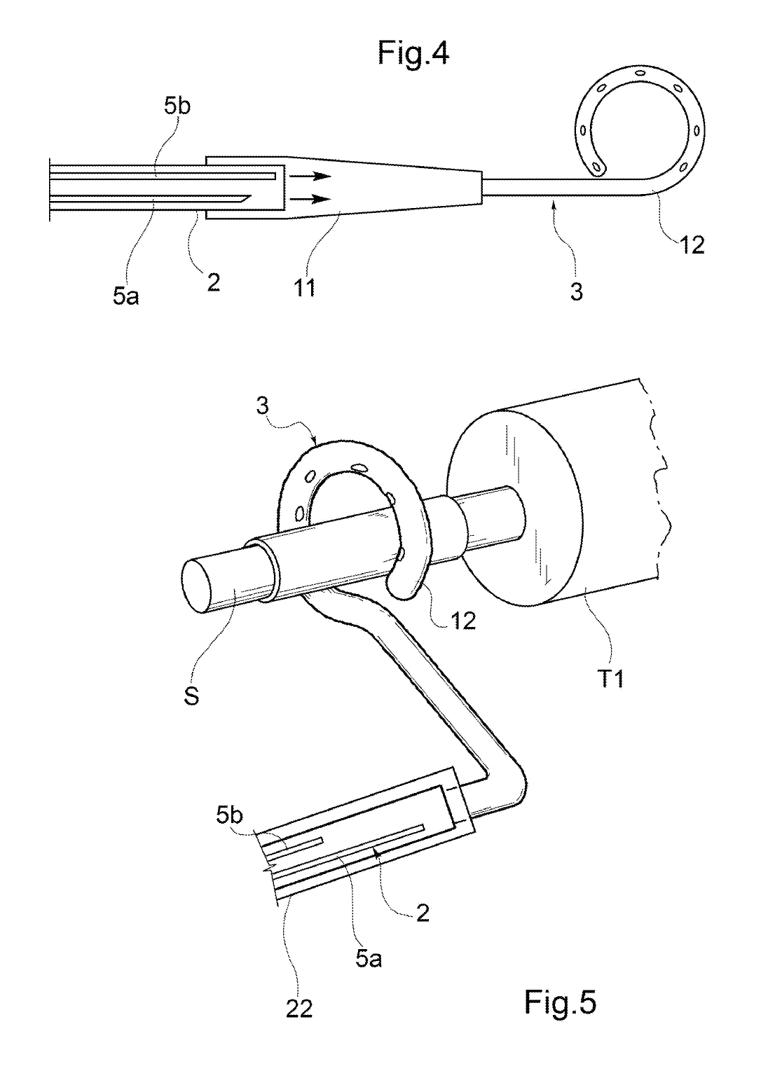

FIGS. 4 to 6 illustrate different embodiments of the glue-diffusing element of a device for applying glue as described herein.

The proximal extremity of the device for applying a connecting glue to the facing extremities of two portions of tissue to be connected—indicated in its entirety with the reference 1—is destined to be handled by an operator, while the distal extremity is destined to perform the functions of the device.

With reference to FIG. 1, the device 1 for applying glue comprises a first tubular sheath 2 wherein at least one conveying conduit 5 is housed. The conveying conduit 5 has the function of conveying the connecting glue, contained, for example, in a reservoir towards a diffusing element 3 for delivering the connecting glue. The diffusing element 3 is positioned at the distal extremity of the first sheath 2 in fluid communication with the conveying conduit 5. A second protecting sheath 22 will be arranged externally to the first sheath 2 and have the capacity of sliding longitudinally along the first sheath 2 between:
an advanced position, wherein the distal section of the sheath 22 covers the diffusing element 3, so to allow the insertion of the device 1 into a trocar to reach the site of application of the glue inside the human or animal body, without causing damage to the diffusing element 3, and
a retracted position, wherein the distal section of the sheath 22 leaves the diffusing element 3 uncovered, allowing it to perform its glue application function.

The proximal extremity of the device 1 is connected to a handle 9 which can assume different shapes, for example pistol or syringe, the function of which is that of controlling delivery of the glue.

In the exemplified case, the handle 9 has a syringe shape allowing the actioning of two pistons 7a, 7b destined to control the delivery of the connecting glue. In this case, it is assumed that the glue is constituted by two components contained in separate sections 4a, 4b of the reservoir 4. In this embodiment, the first sheath 2 will thus house two conveying conduits 5a and 5b for the delivery of the two components of the connecting glue (see FIGS. 2 and 4).

Downstream of the reservoir 4 the device 1 is provided with a collector 6 traversed by the conveying conduit 5. A dispenser of aeriforms under pressure (not illustrated), preferably carbon dioxide or medical air, is connected to the collector 6 in fluid communication by means of a connector 8 for nebulising the connecting glue onto the application site.

The first sheath 2 is connected to the diffusing element 3 by means of a fitting 11 having the function of mixing the connecting glue with the pressurised aeriform for nebulising the glue onto the application site.

The entire device 1 may have a number of shapes and sizes depending on use.

For laparoscopic surgery, the first sheath 2, the second protecting sheath 22 and the conveying conduits 5 may have notable length for reaching tissues inside the human or animal body. Furthermore, the first sheath 2, the second protecting sheath 22 and the conveying conduits 5 may assume a curved shape along their longitudinal axis. Furthermore, the first sheath 2, the second protecting sheath 22 and the conveying conduits 5 may be constituted by a rigid, semi-rigid or flexible material.

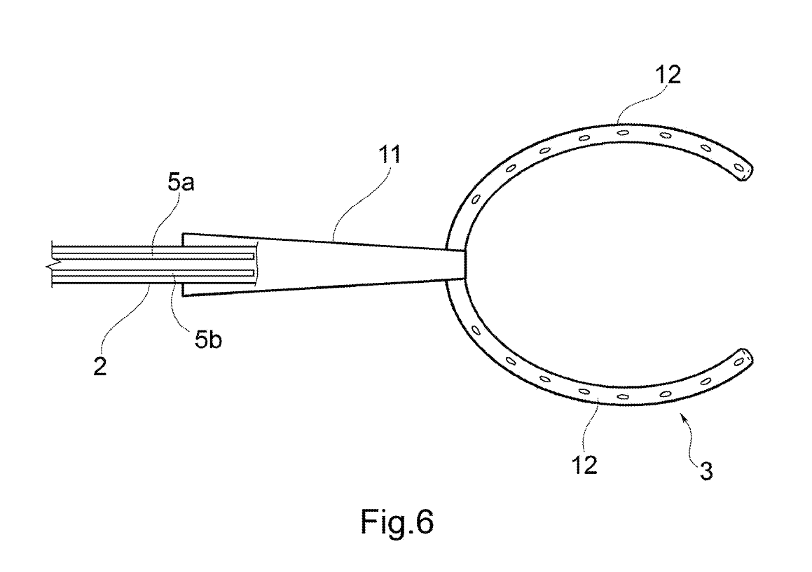

The diffusing element 3 has, in the illustrated embodiments, the shape of a tubular conduit 12 shaped according to a general hooked or curled conformation, folded or not (see FIGS. 4 and 5) or of two or more portions of tubular conduit 12 having a more or less extended arched trend (see FIG. 6) or even a tubular conduit 12 having a substantially linear conformation (not illustrated). The diffusing element 3 can also be provided with a joint to allow assumption of a folded conformation with an angle comprised between about 45° and about 135°, preferably about 90° (see FIG. 5) for easier application of the glue to the facing tissue portions to be connected.

The diffusing element 3 may be constituted by a material with shape memory, so that when the second protective sheath 22 is retracted towards the proximal extremity of the device 1 by the operator, the diffusing element 3 is exposed to the outside environment and may assume its original hooked or curled conformation, folded or not (see FIGS. 4 and 5), or a more or less extended arched trend (see FIG. 6).

Alternatively, the diffusing element 3 may be constituted of a flexible material, for example plastic, with no specific conformation, which is assumed by means of the actioning by the operator of at least one control rod (not illustrated) housed inside the first sheath 2 actionable by the operator through a commander (not illustrated) positioned on the handle 9. The diffusing element 3 can, therefore, be led to assume a hooked or curled conformation, folded or not (see FIGS. 4 and 5), or a more or less extended arched trend (see FIG. 6).

The diffusing element 3, preferably closed at its distal extremity, is provided with diffusion openings 10, extending laterally with respect to the longitudinal trajectory of the diffuser.

Both in the hooked or curled shape (FIGS. 4 and 5), and in the shape with several arched portions (FIG. 6), the above-said diffusing openings 10 are found distributed along an ideal circular or substantially circular trajectory. Being located on opposite sides of the structure of the diffuser the opening can, therefore, diffuse the connecting glue in opposite directions so to apply it onto the facing portion of the tissue to be connected, for example in anastomosis.

It will be appreciated that in all the embodiments illustrated in FIGS. 4 to 6, the diffuser 3 has an open structure, which allows it to deploy, locating it around a stem S of a mechanical suturing machine SM, as illustrated in FIG. 2, so that the connecting glue can be applied on both portions of tissue T1, T2 to be connected. Thus an evident synergy is realised between different devices by means of the advantageous structure of the diffuser 3 which is in this way capable of cooperating with the mechanical suturer SM.

FIG. 3 illustrates a termino-terminal intestinal anastomosis, wherein the two trunks of lumen T1 and T2 have been anastomosed with the mechanical suturer SM applying suture stitches P following the application of a connecting glue C, which can be appreciated as being present on the muscular layer TM and on the mucosal layer TSM, where the molecular reactions take place among thrombin, fibrinogen, fibronectin and factor XIII that are the basis of the healing of the tissues T1 and T2, and only partially on the serous layer TS.

Naturally, the details and embodiments may vary, even appreciably, with reference to what has been described and illustrated, without departing from the field of protection of the present invention as defined by the annexed claims.

1.-10. (canceled)

11. A device for applying a connecting glue to the facing extremities of two portions of tissue to be connected through enteric anastomosis performable with a mechanical suturer, the device comprising a diffusing element suppliable with the glue and interposable between the two portions of tissue to be connected, the diffusing element being provided with at least two openings for bilateral delivery of the glue towards the two portions of tissue to be connected,
wherein said diffusing element is connected in fluid communication to a first sheath,
wherein said device is connected to a dispenser of aeriforms under pressure for delivering said aeriform inside the first sheath for nebulising the connecting glue delivered by the diffusing element to the portions of tissue to be connected, and
wherein said diffusing element has a deployable structure for cooperating with said mechanical suturer.

12. The device according to claim 11, wherein said diffusing element comprises at least one tubular conduit.

13. The device according claim 12, wherein said at least two diffusion openings extend laterally with respect to said at least one tubular conduit.

14. The device according claim 12, wherein said at least one tubular conduit has a hooked or curled conformation.

15. The device according claim 13, wherein said at least one tubular conduit has a hooked or curled conformation.

16. The device according claim 12, wherein said at least one tubular conduit has an arched conformation.

17. The device according claim 13, wherein said at least one tubular conduit has an arched conformation.

18. The device according to claim 11, wherein said diffusing element has an open structure.

19. The device according to claim 11, wherein said diffusing element is constituted of a material selected from: a material with shape memory, a flexible material.

20. The device according to claim 11, wherein said first sheath is susceptible of housing at least one conveying conduit to convey the glue to the diffusing element.

21. The device according to claim 11, wherein when said diffusing element is constituted of a flexible material, said diffusing element is coupled to a control rod housed inside the first sheath, said control rod allowing the diffusing element to assume a hooked or curled conformation or an arched conformation.

22. The device according to claim 20, wherein when said diffusing element is constituted of a flexible material, said diffusing element is coupled to a control rod housed inside the first sheath, said control rod allowing the diffusing element to assume a hooked or curled conformation or an arched conformation.

23. The device according to claim 11, wherein said device also comprises a second protective sheath destined to house the first sheath and the diffusing element inside of it, said protecting sheath being slidable longitudinally along the first sheath, between:
an advanced position, wherein the distal section of said second protecting sheath covers said diffusing element, and
a retracted position wherein the distal section of said second protective sheath leaves said diffusing element uncovered.

24. A device for applying a connecting glue to the facing extremities of two portions of tissue to be connected through enteric anastomosis performable with a mechanical suturer, the device comprising:
i) a diffusing element suppliable with the glue and interposable between the two portions of tissue to be connected, and
ii) a dispenser of aeriform under pressure,
wherein said diffusing element is provided with at least two openings for bilateral delivery of the glue towards the two portions of tissue to be connected,
wherein said diffusing element is connected in fluid communication to a first sheath,
wherein said dispenser of aeriforms under pressure delivers said aeriform inside the first sheath so that the connecting glue delivered by the diffusing element is nebulised to the portions of tissue to be connected, and
wherein said diffusing element has a deployable structure for cooperating with said mechanical suturer.
